(19) United States
(12) Patent Application Publication
(10) Pub. No.: US 2018/0338911 A1
(43) Pub. Date: Nov. 29, 2018

(54) Eye Drops

(71) Applicants: David B. Welch, Winnetka, IL (US); Colman R. Kraff, Highland Park, IL (US)

(72) Inventors: David B. Welch, Winnetka, IL (US); Colman R. Kraff, Highland Park, IL (US)

(21) Appl. No.: 15/771,935

(22) PCT Filed: Oct. 26, 2016
(86) PCT No.: PCT/US16/58812

§ 371 Date: Apr. 27, 2018

Related U.S. Application Data
(60) Provisional application No. 62/247,395, filed on Oct. 28, 2015.

(51) Int. Cl. A61K 9/00; A61K 9/08; A61K 47/32; A61K 47/34; A61P 27/04

(52) U.S. Cl. CPC A61K 9/0048; A61K 9/08; A61P 27/04; A61K 47/34; A61K 47/32

(57) ABSTRACT

A composition and method for applying the composition topically to an eye for improving corneal and surface hydration and reducing blue light neuro-stimulation, especially during the several hours preceding sleep. In one embodiment, the composition combines an artificial tear with fluorescein sodium. The artificial tear may include carboxymehylcellulose sodium, polyvinyl alcohol, hydroxypropyl cellulose and glycerin. In another embodiment, the composition comprises synthetic human melanin combined with fluorescein sodium in an aqueous solution to reduce ocular UV light and HEV light exposure.

### RELATED APPLICATION

This application claims the benefit of U.S. provisional patent application Ser. No. 62/247,395 filed Oct. 28, 2015, the entirety of which is hereby incorporated by reference.

### FIELD OF THE INVENTION

This invention relates to optical solutions and, more particularly, eye drops for inhibiting the suppression of melatonin from viewing light emitting devices and eye drops for filtering ultraviolet and/or high energy visible light.

### BACKGROUND OF THE INVENTION

While most people consider optical radiation to emanate from the sun, the portion of the electromagnetic spectrum that makes up optical radiation also may be produced by lights and other artificial sources. Optical radiation includes UV light (from 100-400 nm), visible light (400-700 nm) and infrared radiation (760-10,000 nm). UV light includes ultraviolet A (“UVA”, from about 320-400 nm), ultraviolet B (“UVB”, from about 290-320 nm) and ultraviolet C (“UVC”, from about 400-500 nm).

Getting an adequate amount of quality sleep is generally considered an important factor in maintaining one's health. Over the last half century, there has been a significant decline in average sleep duration and quality, resulting in adverse consequences on the general health of the population. According to studies cited by the Centers for Disease Control and Prevention, about 20% to 30% of adults have a sleep disorder. One factor that has played a role in the declining quality and quantity of sleep is the increased use of light-emitting screens and other devices, including television, video and computer screens, as well as e-readers. A recent study indicated that 90% of adults used such devices within one hour of going to bed at least a few times a week. Evidence has shown that the high energy visible light (i.e., the light spectrum between 400 nm and 500 nm) emitted from these devices is one of the causes for disruption in sleep patterns. The high energy visible light spectra includes blue light (between 450 nm and 495 nm). Furthermore, under the Energy Independence and Security Act of 2007 requirement that screw-based light bulbs use fewer watts, starting in 2020, most light bulbs must be 60% to 70% more efficient than a standard incandescent bulb. This has and will result in an increased use of LED bulbs. As LED white lights are a blend of the primary light colors re, green and blue, use of LED lights will increase the amount of high energy visible light (“HEV”) exposure.

Prolonged viewing of light-emitting devices also can lead to computer vision syndrome which can have a variety of symptoms including, among others, headaches, blurred vision, fatigue and eye strain. In addition, staring at a light emitting device reduces the amount of blinking, which leads to irritated and dry eyes, and can cause excessive, unfiltered blue-light neuro-stimulation.

Retinal photoreceptive ganglion cells, which are neurons in the retina of the eye, act to assist one's day/night circadian clock. These cells contain the photopigments, melanopsin and cryptochrome, which are most active in the blue light spectrum, and regulate the release or suppression of the sleep promoting hormone melatonin. As a result, the receipt or introduction of blue light and other high energy visible light from the various light emitting devices actively reduces or suppresses the amount of melatonin, and affects the human biologic clock. Studies have shown that the most potent wavelengths for suppressing melatonin synthesis fall primarily within the blue light spectra (446 nm to 477 nm).

A recent study by researchers at Brigham and Women's Hospital in Boston, Massachusetts found that using an e-reader in the period of time before bed resulted in a decreased amount of melatonin, a delay in the time to fall asleep, and less time spent in rapid-eye-movement (REM) sleep. Such sleep reduction and deficiency can have an adverse impact on one's overall health, safety, performance, alertness and natural circadian clock. Recent evidence links the long term suppression of melatonin secretion from nocturnal light exposure to an increased risk of obesity, diabetes, breast cancer, colorectal cancer, and prostate cancer.

Several devices, including blue-light-blocking intraocular lens implants implanted during cataract surgery and blue-light-blocking spectacles, have been utilized to decrease the amount of blue light received and have been shown to have a beneficial effect on sleep quality. However, there is a need for a product that is easy to use without requiring surgery or having to utilize eye glasses or a separate device.

In addition, while UV light does not provide useful vision, it can harm the retina through photo-chemical, photo-thermal and photo-mechanical mechanisms. Among other things, the cornea and the lens absorb, and may propagate UVA and UVB radiation towards the retina.

Furthermore, UV and HEV environmental light exposure may cause and accelerate age-related muscular degeneration (“AMD”). AMD affects 30 to 50 million people worldwide and is the major global cause of irreversible blindness. In addition, UV light has been implicated as a causative etiologic factor for conjunctival pterygia, uveal melanomas and corneal epithelial injury (desiccation/“sun blindness”).

Furthermore, sun exposure may be a risk factor for the development of nuclear cataracts, which currently affect about 20 million Americans and is projected to affect 50 million Americans in the next few decades. While the effects of cataracts are generally reversible, correcting them requires surgery.

While sunglasses, soft and hard contact lenses and intraocular lens implants are available that absorb/filter UV light, individuals do not always have access to these lenses and they are not always worn indoors, especially at night.

Melanin is a complex polymer that is present in most organisms and is responsible for determining skin and hair colors in people. Melanin in the skin acts to absorb and dissipate absorbed ultraviolet radiation. However, while humans have melanin in their bodies, it is often not enough to protect them from damage from exposure to UV light or HEV light. In particular, aging of the human eye is associated with up to 80% loss of ocular melanin, thereby exposing the eye to increased potential damage from UV light and HEV light.

### SUMMARY OF THE INVENTION

The present invention provides a high energy visible light (including blue-light) blocking eye drop and a method for applying the eye drop topically, for example, during the evening prior to working on an LE-screen or e-book before going to bed. The blue-blocking agent would be combined with an artificial tear drop, with the additional benefit of increasing ocular surface hydration. This composition will fill the practical need for an effective and safe topical ophthalmic pharmaceutical composition to manage evening computer vision symptom and blue light neuro-stimulation.

The resulting reduction of high energy visible light transmission to the specialized retinal photoreceptor ganglion cells from using the eye drops will re-synchronize toward the norm the appropriate neuro-stimulation to the specialized retinal photoreceptive ganglion cells. This effectively resets the entrainment from artificial enhanced light to a more natural and physiologic light stimulus.

The composition will safely and practically address the need for ocular surface hydration with a high energy visible light-blocking topical pharmaceutical agent. In one embodiment of the invention, the composition will include fluorescein sodium 0.25% and an artificial tear. The artificial tear may include carboxy-methylcellulose sodium 1%, polyvinyl alcohol, hydroxyl-propyl methylcellulose and glycerin.

Another embodiment of the invention includes a composition comprising synthetic human melanin in the range of 0.001% to 0.1% by concentration, which is soluble at a physiologic pH and at a temperature range of 0° C. to 100° C., combined with fluorescein sodium in the range of 0.25% to 2.0% by concentration in an aqueous solution of polyvinyl 1.4% and povidone 0.6%.

In operation, in order to limit the effects of viewing light emitting devices, particularly, in the few hours before bed, the composition may be applied topically in the form of one or more drops to the eyes to limit the effects of blue light neuro-stimulation. The eye drops may also by applied at other times during the day to reduce the eye's exposure to UV light and HEV light to reduce the development of age related cataracts, ocular malignant melanoma and AMD.

### DETAILED DESCRIPTION OF THE INVENTION

While the present invention is susceptible of embodiment in many different forms, there is herein described in detail, several specific embodiments, with the understanding that the present disclosure is intended as an exemplification of the principles of the present invention and is not intended to limit the invention to the embodiments illustrated.

In one embodiment of the present invention, the composition comprises the combination of a topical ophthalmic tear supplement with an effective amount of fluorescein sodium. Fluorescein sodium, a diagnostic and disclosing agent, has a molecular formula of C₂₀H₁₀Na₂O₅. One current use of fluorescein sodium is a one-time ophthalmic application to view damaged areas of the eye surface in connection with, among other things, detecting dry eyes, corneal abrasions, ulcers or inflammations, measuring pressure in the eye or fitting a person for contact lenses. In one embodiment, the composition includes fluorescein sodium 0.25% by concentration.

While a variety of different topical lubricants, aqueous solutions or artificial tears may be used, one embodiment includes the following active ingredients, inactive ingredients and preservative:

### Active Ingredients

1) Carboxymethyl cellulose sodium 1%
2) Polyvinyl alcohol 1.4%
3) Hydroxypropyl methylcellulose
4) Hydroxypropyl cellulose
5) Hyaluronic acid
6) Glycerin 0.6%
7) Povidone 0.6% or propylene glycol 0.6%
8) Dextran 0.1%
9) Polyethylene glycol 400
10) Polysorbate hypromellose

### Preservative

1) Chlorobutanol 1%

### Inactive Ingredients

1) Boric acid
2) Hydroxyalkphosphonate
3) Purified water
4) Sodium alginate
5) Sodium borate
   The preservative is used to prevent growths of microorganisms. Other known preservatives that may be used include, but is not limited to, BAK.

It is also appreciated that other forms and varieties of artificial tears may be utilized, including: those that integrate natural polymers (e.g., methylcellulose derivatives) and synthetic polymers (e.g., polyethylene glycol, polyvinyl alcohol, povidone, carbopol, polyguar and HP guar) into artificial tear formulations, which are utilized in products such as, but not limited to, Systane (Alcon) and Refresh Optive (Allergan); hyaluronic acid tears, such as those sold under the brand Blink Tears (Abbott Medical Optics); lipid oil-in-water nano-emulsions such as, but not limited to, Soothe XP (Bausch+Lomb), Systane Balance (Alcon) and Retaine (Ocusoft).

The composition may be placed in sterile containers, for example, 0.4 ml single-use containers that are meant to be for preservative free use by consumers.

Application of the composition of the artificial tear and fluororescein sodium is to be done topically by applying one or more drops to the surface of the eye. In order to limit the effects of blue light neuro-stimulation from light emitting device that may interfere with the quality and quantity of one's sleep, the composition may be applied a few hours before a user desires to go to bed. It is also appreciated that the composition may be used to reduce the alerting effects from other high energy visible lights (i.e., those lights between 400 nm and 500 nm on the light spectrum).

The composition may also comprise synthetic human melanin combined with fluorescein sodium. In one embodiment, the composition may include synthetic human melanin in the range of 0.001% to 0.1% by concentration, which is soluble at a physiologic pH and at a temperature range of 0° C. to 100° C., combined with fluorescein sodium in the range of 0.25% to 2.0% by concentration in an aqueous solution of polyvinyl 1.4% and povidone 0.6%. It is further appreciated that the concentration levels of fluorescein and melanin are not limited to this embodiment.

In one embodiment, the melanin has a molecular weight greater than 10,000 kilodaltons, and can be produced by combining dopachrome and an appropriate enzyme, or by incubating 5,6-dihydroxyindole-2-carboxylic acid alone or with 5,6-dihydroxyindole, or with 3-amino-tyrosine. An example of a suitable synthetic melanin is MelaSyn, which is available through Nanotherapeutics of Alachua, Fla. This product may be combined with a topical ophthalmic tear supplement or another aqueous solution such as one including polyvinyl alcohol and povidone. The product is indicated for topical ophthalmic application during the several hours prior to bedtime similar to the topical fluorescein supplement in order to reduce by absorption excess ambient room light and high energy visible (HEV) light, both of which are known to suppress the onset of melatonin synthesis, the duration of melatonin production, and significantly contribute to delayed sleep-phase disorders.

The composition may also be applied topically during the day to provide protection to the eyes by reducing ocular UV light and HEV light exposure. After application, the synthetic melanin eventually will drain with the tear film into the nasolacrimal system and subsequently into the gastrointestinal tract. Accordingly, the topical solution may be reapplied every few hours, or as needed, when encountering continued exposure to UV and HEV light.

Many modifications and variations of the present invention are possible in light of the above teachings. It is therefore to be understood that within the scope of the appended claims, the invention may be practiced other than as specifically described. Various modifications, changes and variations may be made in the arrangement, operation and details of performing the various steps of the invention disclosed herein without departing from the spirit and scope of the invention. The present disclosure is intended to exemplify and not limit the invention.

1. A method for inhibiting the suppression of melatonin in a person exposed to significant levels of emitted light prior to bedtime, consisting of the steps of:
administering eye drops containing a topical lubricant combined with an effective amount of synthetic soluble melanin in the evening prior to bedtime directly onto the person's eye; and
after the step of administering the eye drops, viewing by the same person, of light from a light-emitting screen of an electronic device for a duration of at least one hour to, in turn, inhibit the suppression of melatonin in the brain of the same person.

2. The method of claim 1 wherein the topical lubricant comprising an aqueous solution.

3. The method of claim 2, wherein the aqueous solution comprises an artificial tear.

4. (canceled)

5. (canceled)

6. (canceled)

7. The method of claim 1 wherein the eye drop further comprises synthetic soluble melanin in the range of 0.001% to 0.1% by concentration.

8. The method of claim wherein the eye drop comprises synthetic soluble melanin 0.1% by concentration.

9. The method of claim 2 wherein the aqueous solution comprises polyvinyl 1.4% and povidone 0.6%.

10. A composition for optical application to inhibit the suppression of melatonin caused by viewing emitted light, the composition comprising:
a topical lubricant; and
a high energy visible light blocking material comprising synthetic soluble melanin.

11. The composition of claim 10, wherein the topical lubricant comprises an aqueous solution.

12. The composition of claim 11, wherein the aqueous solution comprises an artificial tear.

13. (canceled)

14. (canceled)

15. The composition of claim 10 wherein the high energy visible blocking material comprises synthetic soluble melanin in the range of 0.001% to 0.1% by concentration.

16. The composition of claim 10 wherein the high energy visible blocking material comprises synthetic soluble melanin 0.1% by concentration.

17. The composition of claim 10 wherein the topical lubricant comprises an aqueous solution of polyvinyl 1.4% and povidone 0.6%.

18. A method for inhibiting the exposure of ultraviolet and high energy visible light to the eyes of a person exposed to emitted light, consisting of the step of administering eye drops containing a topical lubricant comprising an effective amount of synthetic soluble melanin.

19. The method of claim 18 wherein the eye drops comprise the synthetic soluble melanin in the range of 0.001% to 0.1% by concentration.

20. The method of claim 19 wherein the topical lubricant comprises an aqueous solution of polyvinyl 1.4% and povidone 0.6%.

21. A composition for optical application to inhibit damage to the eye caused by the exposure to UV and HEV lights by viewing emitted light, the composition comprising:
a topical lubricant; and
an ultraviolet and high energy visible light blocking material comprising synthetic soluble melanin.

22. The composition of claim 21, wherein the topical lubricant comprises an aqueous solution.

23. The composition of claim 22, wherein the aqueous solution comprises an artificial tear.

24. The composition of claim 21, wherein the high energy visible light blocking material comprises the synthetic soluble melanin in the range of 0.001% to 0.1% by concentration.
